(19) United States
(12) Patent Application Publication
(10) Pub. No.: US 2009/0171255 A1
(43) Pub. Date: Jul. 2, 2009

(54) Apparatus and method for ultrasound treatment

(76) Inventors: Andrey Rybyanets, Yoqneam (IL); Allan C. Entis, Tel Aviv (IL)

Correspondence Address: EMPK & Shiloh, LLP;c/o Landon IP, Inc., 1700 Diagonal Road, Suite 450, Alexandria, VA, 22314, US

(21) Appl. No.: 12/005,285

(22) Filed: Dec. 27, 2007

(51) Int. Cl. A61N 7/02

(52) U.S. Cl. 601/3

(57) ABSTRACT

Apparatus for lysing adipose tissue and/or cellulite comprising: at least one transducer controllable to transmit ultrasound; and an adapter configured to couple ultrasound transmitted by the at least one transducer to a region of a patient's skin comprising and/or overlaying a layer of adipose tissue and/or cellulite; wherein the adapter orients the transmitted ultrasound relative to the layer of adipose tissue and/or cellulite so that it tends to propagate along a region of the layer.

### FIELD

The invention relates to methods and apparatus for performing acoustic procedures on tissue.

### BACKGROUND

Various methods are known for delivering and coupling acoustic energy to a region of tissue to perform a diagnostic and/or therapeutic and/or cosmetic procedure on a patient's tissue. Among such procedures are for example, non-invasive assaying of blood analytes, drug delivery by phonophoresis, lithotripsy, tissue ablation and lysis of fat cells for cosmetic removal of adipose tissue.

For many types of therapeutic and/or cosmetic acoustic applications, such as for example lithotripsy, tissue ablation and lysis noted above, sufficient acoustic energy must be delivered to a tissue region to destroy and remove tissue in the region. Generally, the acoustic energy is delivered by focusing at least one beam of relatively intense ultrasound on the region. The high intensity, focused ultrasound, conventionally referred to by the acronym “HIFU”, may be used to generate various thermal and mechanical effects on tissue that include local heating of tissue and/or cavitation that disrupts and destroys the tissue. Tissue raised to and maintained at a temperature above about 42° C. rapidly dies and mechanical stresses generated by cavitation breach and tear cell membranes of the tissue.

However, it is often difficult to control high-energy focused ultrasound to satisfy constraints that may be required to perform various procedures for which it is intended. For example, HIFU beams are often focused to a relatively small volume of tissue and can require a relatively large dwell time at the focal volume to destroy tissue therein. Typically, a focal volume of a HIFU beam is substantially contained within a prolate ellipsoid. For a frequency of ultrasound equal to about 200 kHz, the ellipsoid has a long axis of about 15 mm along a direction of propagation of the beam and a cross section that bisects the long axis and is perpendicular to the propagation direction having a radius of about 6 mm. For frequency of about 1 MHz, the long axis is about 3 mm and the cross section has a radius of about 1.5 mm. In general, the focal volume has lateral diameter of approximately 1 wavelength and a length of between about 2-3 wavelengths. (Boundaries of the focal volume are assumed to be in regions where acoustic intensity is attenuated by about 6 dB.) Treating an extended region of tissue with HIFU can therefore often be a relatively tedious task that requires a relatively long time to perform. As a result, various techniques have been proposed and/or used for expanding a useful focal volume of HIFU beams and for electronically and/or mechanically scanning the beams to treat relatively large tissue volumes.

However, control of HIFU beams to deliver effective acoustic energy that is spatially relatively homogenous over an extended tissue volume that is a desired target for treatment and that does not adversely affect non-target tissue can be problematic. Often configurations of extended focal volume HIFU beams exhibit “hot spots” that limit therapeutic and/or cosmetic use of the beams. And, ultrasound that is propagated into the body so that it is substantially focused in a desired region generally propagates through and past the focal region and is incident on organs and/or body features for which the ultrasound is not intended.

For example, adipose tissue generally resides in the subcutaneous layer of the skin and is located in a region from about a few mm to a few tens of mm below the skin surface. In procedures for tissue ablation and lysis of fat cells for cosmetic removal of adipose tissue, ultrasound focused to fat tissue below the skin may propagate beyond the adipose tissue, impinge on, and damage internal organs and body features lying below the subcutaneous layer. If the ultrasound is being used to treat belly fat, the ultrasound may for example, be incident on the liver. If the ultrasound is used to treat cellulites in the hip region, the ultrasound may be incident on and reflected from bone tissue below the skin. The reflected ultrasound can interfere with the ultrasound propagated into the body to treat the cellulites and generate a standing acoustic wave having intensity at or near the skin surface that can damage the skin.

F. L. Lizzi et al in an article entitled “Asymmetric Focussed Arrays for Ultrasonic Tumor Therapy” describe using “spherical cap transducers with segmented rectangular electrodes” to provide HIFU beams useable to produce lesions with elliptical cross-sections. By phasing excitation of pairs of rectangular electrodes, undesired axial regions of high acoustic intensity of the beams in planes other than a focal plane of the transducers were suppressed. To treat extended tissue regions, the beams are intended for scanning along a direction substantially perpendicular to the long axes of the elliptical lesions.

US Patent Application Publication US 2005/0154314 to J. U. Quistgaard, entitled “Component Ultrasound Transducer” promulgates objects of the invention of the application as: “to provide a transducer capable of transmitting high intensity ultrasound energy into two or more focal zones simultaneously” and also “to provide for a transducer capable of focusing two or more different frequencies into a single focal zone, or into a group of focal zones.”

In an article entitled “Concentric-Ring and Sector-Vortex Phased-Array Applicators for Ultrasound Hyperthermia” by C. A Cain and S. I. Umemura, concentric ring and radial sector elements of a transducer are excited with various phase configurations to produce concentric circularly symmetric focal rings of acoustic energy for which acoustic energy at a focus along a central axis is “greatly reduced”. Phasing that generates circularly asymmetric elliptic focal regions is also described.

U.S. Pat. No. 6,506,171 S. Vitek and N. Brenner describe a focused ultrasound system that “includes a plurality of transducer elements disposed about and having an angular position with a central axis”. The various sector elements are excited with phases so that “a first on-axis focal zone and a second off-axis focal zone are created”. The figures in the application show that the second off-axis focal zone is characterized by a plurality of focal regions, in each of which acoustic energy focused to the region has a substantially same spatial energy distribution. The plurality of focal regions exhibits an almost perfect rotational rosette-like symmetry.

All the above referenced documents are incorporated herein by reference.

### SUMMARY OF THE INVENTION

An aspect of some embodiments of the invention relates to providing apparatus for treating a region of a patient's tissue using ultrasound that provides relatively enhanced localization of ultrasound in the tissue region to be treated. Optionally, the tissue comprises adipose tissue and or cellulite located in or in the neighborhood of the patient's subcutaneous layer.

An aspect of some embodiments of the invention relates to providing apparatus for treating relatively extended regions of a patient's tissue with focused ultrasound for which the focal volume of the ultrasound overlaps a relatively large portion of the tissue region.

An aspect of some embodiments of the invention relates to providing apparatus that orients the direction of propagation of the ultrasound relative to the tissue to be treated to provide improved localization of the ultrasound to the tissue and/or to provide a focal volume of the ultrasound that overlaps a relatively large volume of the tissue.

In an embodiment of the invention, the apparatus comprises at least one ultrasound transducer operable to transmit ultrasound into a patient's body to treat a tissue region of the patient and an adapter that couples the transducer to the patient's skin. Optionally, the adapter comprises an acoustic coupler having shape and acoustic impedance configured to direct ultrasound generated by the at least one transducer so that it enters the body along a direction that improves localization and coverage of the tissue region by the ultrasound.

In an embodiment of the invention, the adapter comprises a skin clamp that holds the patient's skin relative to the at least one transducer so that ultrasound enters the region to be treated along a desired direction. Optionally, the skin clamp is a mechanical clamp having two opposing gripping surfaces that are controllable to selectively be moved toward and away from each other to grasp a portion of skin and tissue underlying the skin and lift up and sandwich the portion between them. When drawn up and sandwiched between the gripping surfaces, a relatively extended region of “target” tissue, optionally, adipose tissue in the subcutaneous layer of the patient's skin and neighborhood thereof is positioned substantially parallel to and along a plane that is substantially parallel to the skin orientation prior to the skin being drawn up and sandwiched between the gripping surfaces.

Hereinafter, position of a region of skin that is not displaced from its normal location on a patient's body is referred to as a “normal” skin position. A plane parallel to and located above a normal position of skin and along which target tissue is to be located after the skin is drawn up in accordance with an embodiment of the invention, is referred to as a “target plane”.

In accordance with an embodiment of the invention, the at least one ultrasound transducer is positioned to transmit focused ultrasound into and through the target adipose tissue from one side to the other side of the sandwiched skin along a direction that is substantially parallel to and coincident with the target plane. Because the ultrasound is transmitted substantially parallel to the target plane, a focal volume of the ultrasound has a long axis that lies substantially in the target plane. Since both the focal volume and a relatively large portion of the adipose tissue are parallel to and lie along the target plane, the focal volume is coincident with a relatively large volume of the adipose issue. In particular, the focal volume is coincident with a generally larger volume of the target tissue than would be the case were the ultrasound transmitted into the patient's body, as in prior art, along a direction substantially perpendicular to the patient's skin, i.e. perpendicular to the normal position of the skin, and thereby substantially perpendicular to the subcutaneous layer. Furthermore, because the ultrasound is transmitted substantially parallel to the target plane, and the target plane is parallel to the normal position of the skin before the skin is drawn up and sandwiched between the gripping surfaces, substantially no acoustic energy from the transmitted ultrasound propagates into the body and is incident on internal organs.

In some embodiments of the invention, the adapter comprises an absorber that absorbs ultrasound transmitted by the at least one transducer that traverses the sandwiched skin portion. The absorber is located on a surface or surfaces of the adapter or the apparatus comprising the adapter on which the ultrasound is incident after traversing the tissue to be treated. The absorber tends to prevent acoustic energy from being reflected from the surface or surfaces and interfering with the incident acoustic energy to generate standing sound waves having relatively high intensity acoustic hot spots that can be damaging to skin and other tissue.

In an embodiment of the invention, the adapter comprises an aspiration chamber operable to draw up and position a portion of skin substantially along a target plane parallel to and located above the skin's normal position. The aspiration chamber comprises at least one outlet through which air may be aspirated from the chamber and is configured to be placed on a region of the patient's skin so that the aspiration chamber is sealed against ingress of air by the skin when air is withdrawn from the chamber through the outlet. When air is aspirated from the chamber, a partial vacuum is formed in the aspiration chamber that draws the skin into the chamber so that a portion of the skin, such as a portion of the skin's subcutaneous layer, is positioned substantially parallel to and coincident with the target plane.

For convenience of presentation, apparatus for treating a region of a patient's tissue using ultrasound that is operable in accordance with an embodiment of the invention to direct ultrasound to the region along a direction that enhances the efficacy of the ultrasound is referred to as a “direction enhanced ultrasound (DEUS) apparatus”, a “DIEUS apparatus” or “DEUS”.

There is therefore provided, in accordance with an embodiment of the invention, apparatus for lysing adipose tissue and/or cellulite comprising: at least one transducer controllable to transmit ultrasound; and an adapter configured to couple ultrasound transmitted by the at least one transducer to a region of a patient's skin comprising and/or overlaying a layer adipose tissue and/or cellulite; wherein the adapter orients the transmitted ultrasound relative to the layer of adipose tissue and/or cellulite so that it tends to propagate along a region of the layer.

Optionally, the apparatus comprises a clamp having first and second panels which is operable to move the first panel towards the second panel to pinch up and clamp the region of skin and adipose and/or cellulite layer between the panels. Optionally, the at least one transducer is located on the first panel and when controlled to transmit ultrasound, transmits the ultrasound in a direction toward the second panel. Optionally, the apparatus comprises an acoustic absorber located on the second panel.

In some embodiments of the invention, the clamp is manually operable to move the first panel towards the second panel to pinch up and clamp the region of skin and adipose and/or cellulite layer.

In some embodiments of the invention, the apparatus comprises a motor or actuator operable to move the first panel towards the second panel to pinch up and clamp the region of skin and adipose and/or cellulite layer.

In some embodiments of the invention, the at least one transducer is controllable to transmit ultrasound in a direction substantially parallel to a portion of the adipose layer and/or cellulite clamped between the first and second panels. Optionally, the at least one transducer is controllable to focus the ultrasound in a focal volume located in the portion of the adipose and/or cellulite layer. Optionally, the at least one transducer is controllable to translate the focal volume in a direction substantially perpendicular to a direction along which the clamp pinches up the region of skin and substantially perpendicular to a direction along which the at least one transducer transmits the ultrasound.

Additionally or alternatively, the at least one transducer is controllable to focus the ultrasound in a focal volume that has an extent substantially equal to an extent of the at least one transducer in a direction substantially perpendicular to a direction along which the clamp pinches up the region of skin and substantially perpendicular to direction along which the at least one transducer transmits the ultrasound. Optionally, the adapter comprises an aspiration chamber into which the region of skin and the adipose and/or cellulite layer can be drawn up by aspirating air from the chamber. Optionally, the at least one transducer is located on an internal surface of the aspiration chamber substantially parallel to a direction along which the region of skin and adipose and/or cellulite layer is drawn up into the aspiration chamber.

Additionally or alternatively, the at least one transducer is controllable to transmit ultrasound in a direction substantially parallel to a portion of the adipose and/or cellulite layer drawn up into the aspiration chamber. Optionally, the at least one transducer is controllable to focus the ultrasound in a focal volume located in the portion of the adipose and/or cellulite layer. Optionally, the at least one transducer is controllable to move the focal volume. Optionally, the at least one transducer is controllable to rotate the focal volume around an axis substantially parallel to the direction along which the region of skin and adipose tissue and/or cellulite is drawn up into the aspiration chamber.

In some embodiments of the invention, the aspiration chamber is cylindrical.

There is further provided, in accordance with an embodiment of the invention, apparatus for lysing adipose tissue and/or celluolite in a patient, the apparatus comprising: at least one transducer controllable to transmit ultrasound; and a wedge shaped transducer having a first surface on which the at least one transducer is located and a second surface through which ultrasound transmitted by the at least one transducer enters the patient's skin; wherein the first and second surfaces are angled with respect to each other by a wedge angle. Optionally, the wedge angle is between about 5° and about 45°. Optionally, the wedge angle is between about 15° and about 35°.

In some embodiments of the invention, the at least one transducer comprises a plurality of transducers configured in a phased array of transducers.

There is further provided, in accordance with an embodiment of the invention, a method of treating adipose tissue and/or cellulite in a patient, the method comprising: drawing a region of the patient's skin and a layer of adipose tissue and/or cellulite comprised in the skin and/or overlaid by the skin away from the body; and transmitting ultrasound through the drawn away skin region and adipose tissue so that it propagates through a portion of the drawn away adipose and/or cellulite layer along a direction substantially parallel to the layer.

Optionally, transmitting ultrasound along a direction substantially parallel to the layer comprises transmitting the ultrasound along a direction substantially perpendicular to a direction along which the skin region is drawn away from the body. Additionally or alternatively, transmitting comprises focusing the ultrasound in a focal volume located in the portion of the adipose and/or cellulite layer. Optionally, the method comprises moving the focal volume. Optionally, moving comprises translating the focal volume along a direction substantially perpendicular to the direction along which the region of skin and adipose tissue and/or cellulite is drawn away from the body. Additionally or alternatively, moving comprises rotating the focal volume along a direction substantially parallel to the direction along which the region of skin and adipose tissue and/or cellulite is drawn away from the body.

### BRIEF DESCRIPTION OF FIGURES

Non-limiting examples of embodiments of the invention are described below with reference to figures attached hereto that are listed following this paragraph. Identical structures, elements or parts that appear in more than one figure are generally labeled with a same numeral in all the figures in which they appear. Dimensions of components and features shown in the figures are chosen for convenience and clarity of presentation and are not necessarily shown to scale.

FIG. 1 schematically shows a DEUS apparatus comprising a skin clamp and operable to lyse adipose tissue in a tissue region of a patient, in accordance with an embodiment of the invention;

FIGS. 2A-2C schematically illustrate the DEUS apparatus shown in FIG. 1 being used to lyse adipose tissue, in accordance with an embodiment of the invention;

FIG. 3A schematically shows a DEUS apparatus comprising an aspiration chamber for holding a region of skin to be treated with ultrasound generated by the DEUS apparatus, in accordance with an embodiment of the invention;

FIGS. 3B and 3C schematically illustrate the DEUS apparatus shown in FIG. 3 being used to lyse adipose tissue, in accordance with an embodiment of the invention;

FIG. 3D schematically shows another DEUS apparatus comprising an aspiration chamber for holding a region of skin to be treated with ultrasound generated by the DEUS apparatus, in accordance with an embodiment of the invention;

FIG. 4A schematically shows another apparatus being used to lyse adipose tissue; and

FIG. 4B schematically shows another DEUS apparatus being used to lyse adipose tissue, in accordance with an embodiment of the invention.

### DETAILED DESCRIPTION

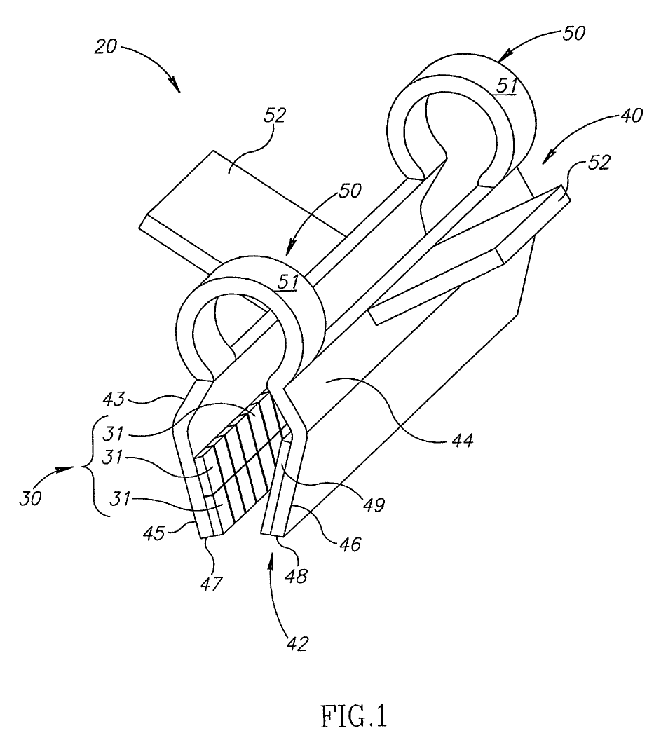

FIG. 1 schematically shows a DEUS apparatus 20 for lysing adipose issue in a patient, in accordance with an embodiment of the invention. DEUS 20 optionally comprises at least one piezoelectric transducer, optionally a phased array 30 of piezoelectric transducers 31 mounted to an adapter 40 and controllable to generate ultrasound. Any of various shaped transducer and phased array configurations known in the art may be used to configure phased array 30. By way of example, phased array 30 comprises two rows of rectangular piezoelectric transducers 31, each of which is closely adjacent to at least two other piezoelectric transducers 31. In accordance with an embodiment of the invention, adapter 40 of DEUS 20 has a skin clamp 42 for gripping a portion of a patient's skin having adipose tissue to be treated with ultrasound generated by phased array 30 DEUS apparatus 20.

Skin clamp 42 optionally comprises two hinge panels 43 and 44 that are hinged together by at least one hinge and are connected respectively to gripping panels 45 and 46 respectively having edges 47 and 48. Panels 45 and 46 are planar in FIG. 1 and figures that follow. In some embodiments of the invention, gripping panels 45 and 46 are other than planar and may, for example, be cylindrical. Piezoelectric transducers 31 of phased array 30 are mounted to gripping panel 45. Gripping panel 46 is optionally mounted with a material 49 that is a relatively good absorber of ultrasound generated by phased array 30. Optionally, gripping panels 45 and 46 are planar.

At least one resilient element comprised in adapter 40 operates to urge gripping panels 45 and 46 toward each other. Optionally, the at least one resilient element comprises two flexure hinges 50. Each flexure hinge 50 optionally comprises a band 51 of resilient material bent into an arc, which operates to elastically urge gripping panels 45 and 46 together. When unstressed, as shown in FIG. 1, flexure hinges 50 maintain gripping panels so that edges 47 and 48 of the gripping panels are relatively close. Each hinge panel 43 and 44 optionally comprises a spreading handle 52. By applying sufficient pressure, optionally manually, to move spreading handles 52 toward each other, band 51 of each flexure hinge 50 is opened up and gripping panels 45 and 46 are moved away from each other.

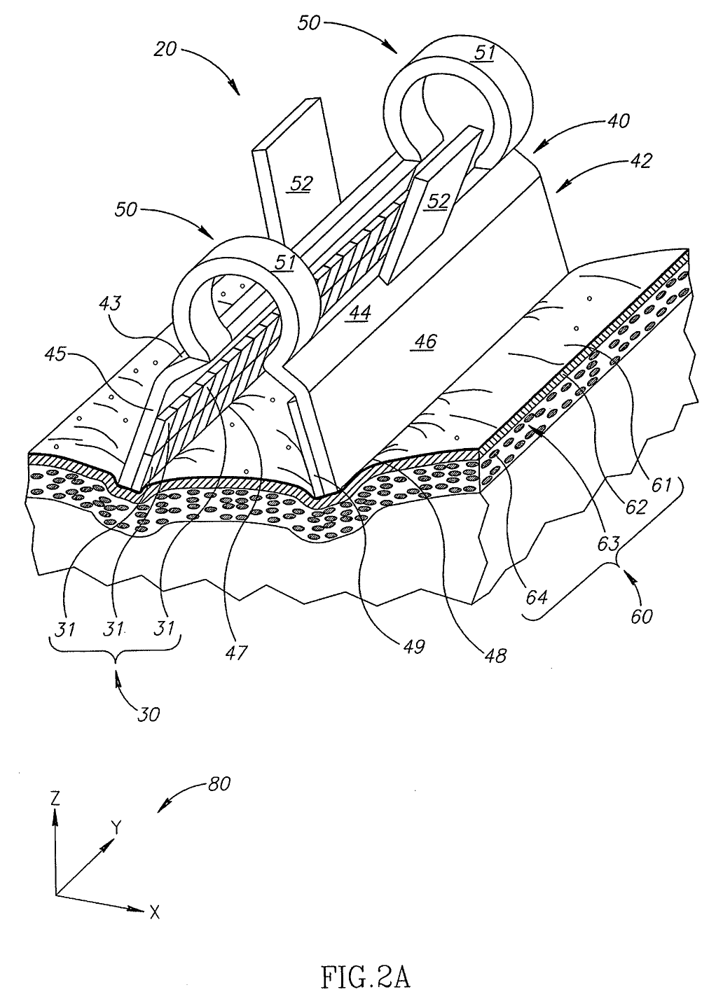

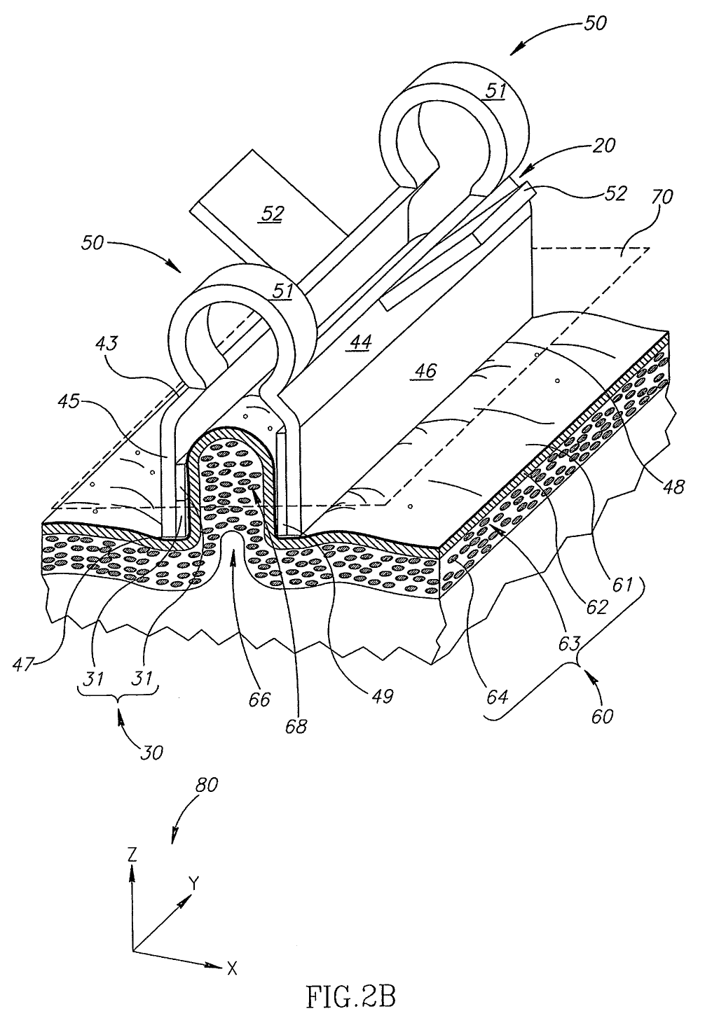

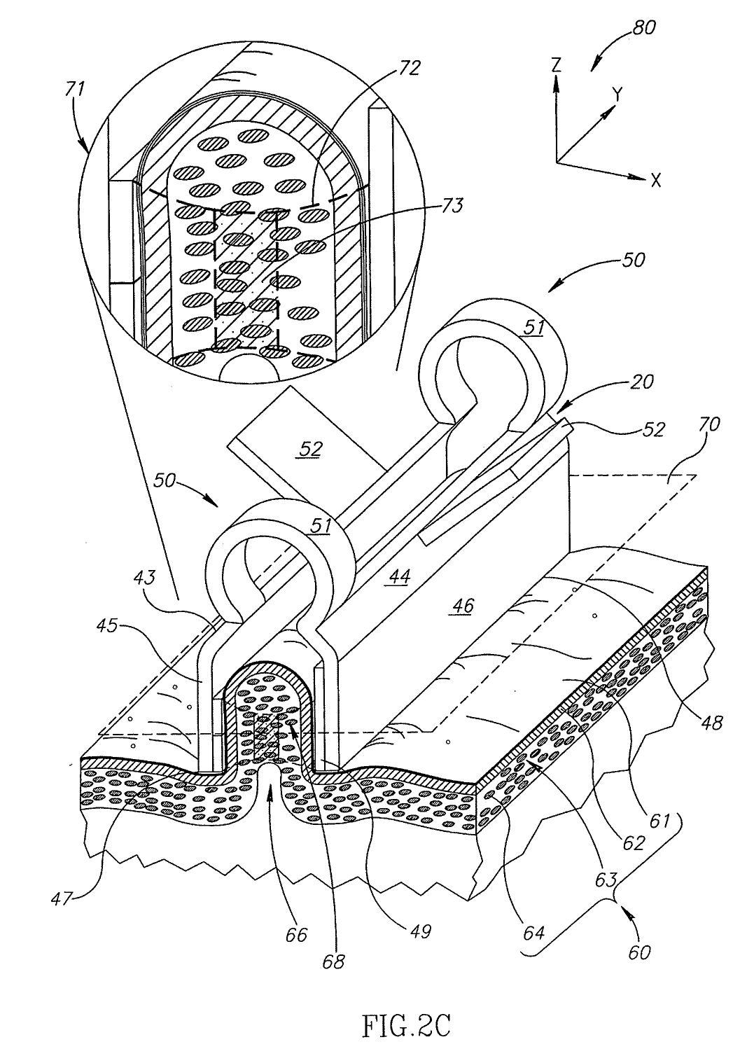

FIGS. 2A-2C schematically illustrate DEUS apparatus 20 being used to lyse adipose tissue in a region 60 of a patient's skin. The figures schematically show a cross section of skin region 60 to exhibit epidermis 61, dermis 62 and subcutaneous layer 63 of the skin. The subcutaneous layer comprises adipose tissue 64. Position and orientation of features of DEUS 20 in FIGS. 2A-2C are referenced relative to a coordinate system 80 when convenient for clarity of presentation. The x-axis and y-axes of coordinate system 80 are respectively perpendicular and parallel to edges 47 and 48 and substantially coplanar with a plane containing edges 47 and 48.

In FIG. 2A, spreading handles 52 are shown pressed together, optionally manually, to displace gripping panels 45 and 46 away from each other and spread apart edges 47 and 48 of the panels. While the gripping panels are spread apart, DEUS apparatus 20 is pressed to skin region 60 so that spread apart edges 47 and 48 are firmly depressed into the skin. When positioned as shown in FIG. 2A, force displacing spreading handles 52 toward each other is released so that flexure hinges 50 urge gripping panels 45 and 46 to come together and “pinch up” a volume of skin 60 and tissue underlying the skin and clamp the pinched up tissue between the gripping panels.

FIG. 2B schematically illustrates a tissue volume 66 pinched up and clamped between gripping panels 45 and 46, in accordance with an embodiment of the invention. When pinched up as shown in the figure, a relatively extended region 68 of subcutaneous layer 63 and its adipose tissue 64 is held along a target plane, schematically indicated by a dashed rectangle 70, of phased array 30.

To lyse adipose tissue 64 in subcutaneous layer 63, phased array 30 is controlled to focus ultrasound in a focal volume, substantially contained within an envelope having a cross section in target plane 70 characterized by relatively large dimensions. In an embodiment of the invention, the cross section has a relatively long extent in target plane 70 in a direction substantially perpendicular to the phased array. Optionally, the focal volume has a relatively long extent parallel to the phased array.

FIG. 2C schematically shows an envelope, shown enlarged in an inset 71, outlined in dashed lines 72 of ultrasound energy generated by phased array 30 and focused along target plane 70 parallel to the xy-plane of coordinate system 80, in accordance with an embodiment of the invention. Along target plane 70, volume 72 has a relatively narrow waste region 73, shown shaded, that is a focal volume of the ultrasound generated by phased array 30. Ultrasound radiated by phased array 30 passes through focal region 73 where it is concentrated to reach a relatively high intensity optionally sufficient to lyse adipose tissue 64 in the region. Ultrasound not absorbed by tissue in focal region 73 propagates on towards acoustic absorber 49 where it is absorbed by the absorber.

In some embodiments of the invention, phased array 30 generates a focal region 73 having an extent parallel to the y-axis that is relatively small compared to a length phased array 30 parallel to the y-axis. For such cases, the phased array is optionally controlled to translate the focal region parallel to the y-axis to treat and optionally lyse adipose tissue 64 along target plane 70 substantially all along the y-axis between gripping panels 45 and 46. By way of example, in FIG. 2C, focal region 73 is shown relatively small and located near one end of clamp 42. In accordance with an embodiment of the invention, phased array 30 is controlled to translate the focal region parallel to the y-axis to the other end of the clamp to lyse adipose tissue along substantially all the length of subcutaneous layer 63 clamped between gripping panels 45 and 46.

Because subcutaneous layer 63 of skin 60 and focal volume 73 have relatively large dimensions parallel to the x-axis along target plane 70, focal volume 73 overlaps a relatively large volume of adipose tissue 64 in the skin. As a result, DEUS 20 is operable to treat and optionally lyse relatively large volumes of the adipose tissue rapidly and efficiently. Furthermore, because ultrasound generated by phased array 30 does not propagate in a direction that intersects internal organs and features of the patient's body, there is relatively little probability that the ultrasound will interact and damage the patient's internal tissues.

In some embodiments of the invention, phased array 30 is controlled to illuminate adipose tissue along target plane 70 with ultrasound having a focal region of relatively high intensity ultrasound whose extent parallel to the y-axis is substantially equal to a full length along the y-axis of the phased array. In such embodiments, adipose tissue lying along plane 70 and having an extent parallel to the y-axis substantially equal to that of the phased array is simultaneously treated with high intensity ultrasound.

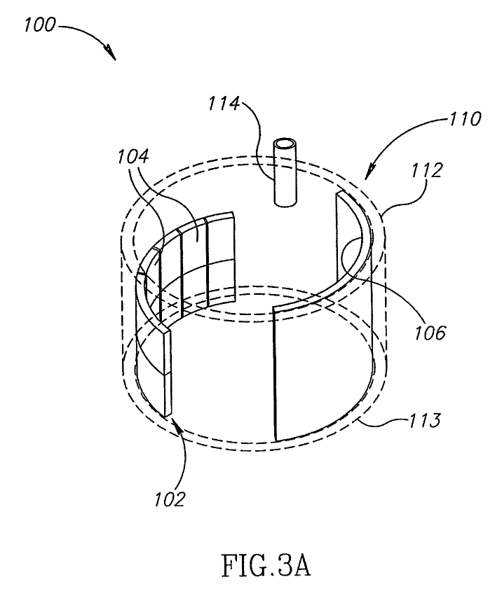

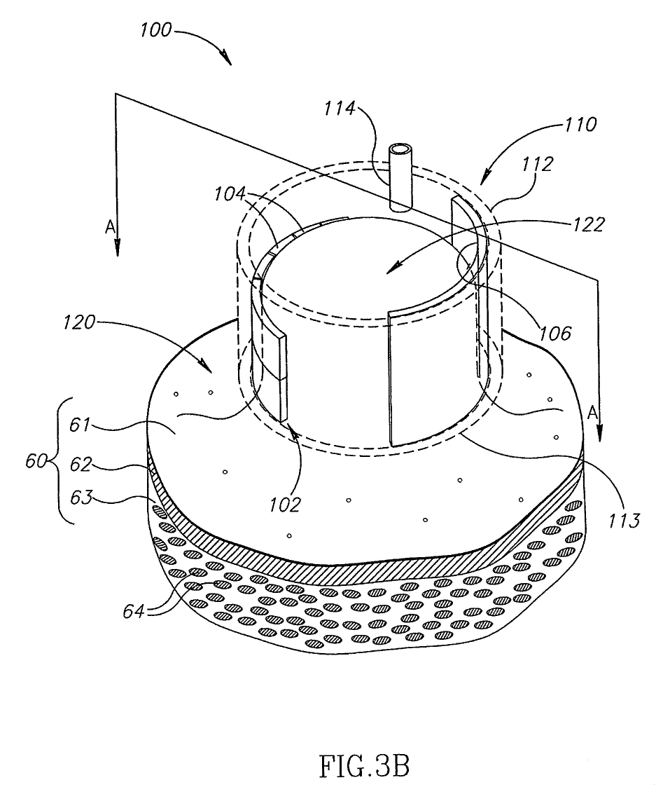

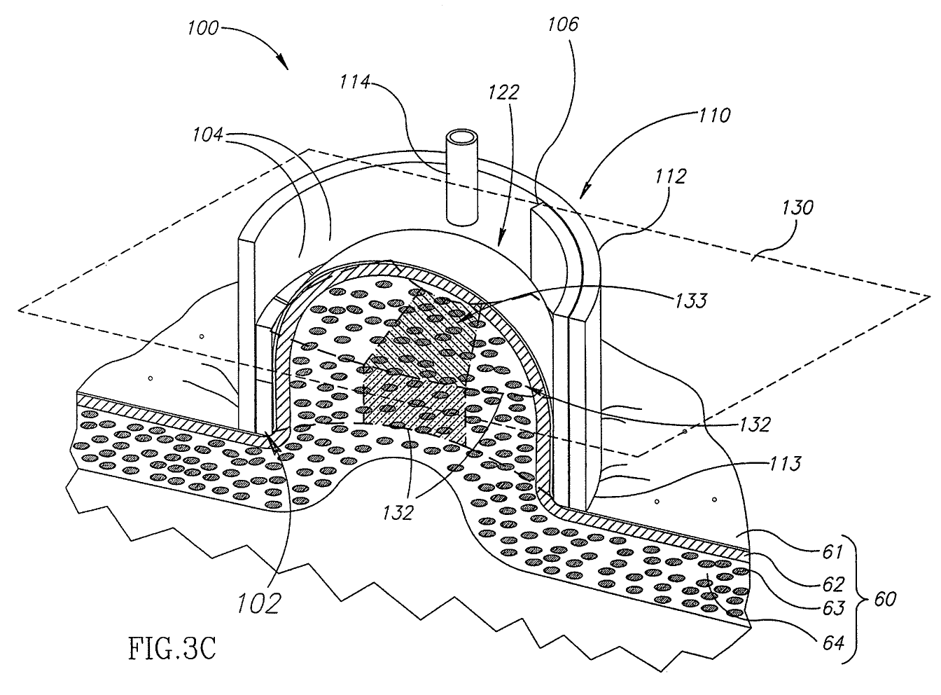

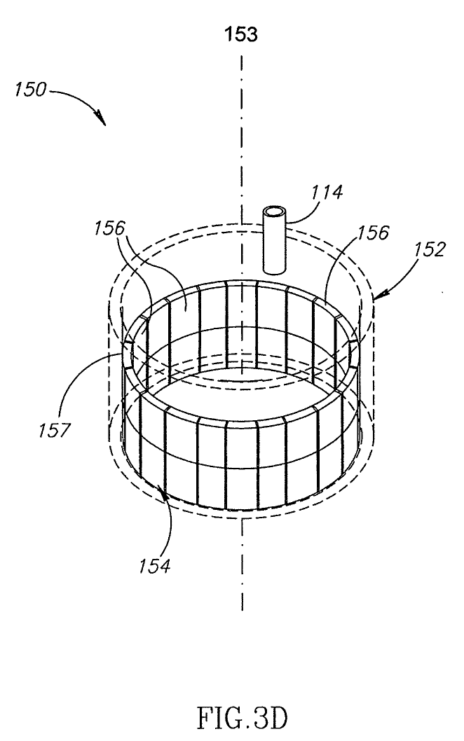

FIG. 3A schematically shows another DEUS apparatus 100 for treating tissue with ultrasound, in accordance with an embodiment of the invention. Optionally, DEUS 100 comprises a phased array 102 of piezoelectric transducers 104 having a cylindrical shape located opposite a cylindrical acoustic absorber 106. The phased array and the absorber are mounted inside an adapter 110 comprising an optionally circularly cylindrical aspiration chamber 112, shown in dashed lines, having a bottom edge 113 and comprising an aspiration outlet 114 through which air in the chamber may be aspirated. Optionally, outlet 114 is configured to be attached to a flexible tube so that air can be aspirated by mouth from chamber 112.

FIGS. 3B and 3C schematically illustrate operation of DEUS 100 to lyse adipose tissue in a region 120 of a patient's skin 60, in accordance with an embodiment of the invention. As schematically shown in FIG. 3B, aspiration chamber 112 is pressed to region 120 to seal edge 113 of the chamber to the skin, and air is aspirated from the chamber via aspiration outlet 114 to draw a portion 122 of the region up and into the chamber.

FIG. 3C schematically shows DEUS 100 and skin portion 122 shown in FIG. 3B partially cutaway along a plane AA indicated in FIG. 3B. Subcutaneous layer 63 of skin portion 122 is located along a target plane 130, and phased array 102 is controlled to focus ultrasound along the target plane so that a focal region of the ultrasound overlaps a relatively large region of subcutaneous layer 63 in accordance with an embodiment of the invention. Dashed lines 132 schematically indicate an envelope of ultrasound focused by phased array 102 along target plane 130, and a focal region of the ultrasound that overlaps subcutaneous layer 63 is indicated by a shaded region 133 of the envelope. Acoustic absorber 106 absorbs ultrasound that passes through focal region 133 and is not absorbed by tissue in skin portion 122.

As in the case of DEUS 20 shown in FIG. 1-FIG. 2C, focal region 133 of DEUS 100 overlaps a relatively large region of adipose tissue 64 in skin portion 122, and DEUS 100 is therefore controllable to lyse a relative large region of adipose tissue relatively rapidly.

Whereas phased array 102 in DEUS 100 extends along a cylindrical arc surface, practice of the invention is not limited to phased arrays covering a circularly cylindrical arc surface. For example, FIG. 3D schematically shows a DEUS 150 comprising an optionally circularly cylindrical aspiration chamber 152 having an axis 153 and a phased array 154 of piezoelectric transducers 156 that cover a complete, inside surface 157 of the aspiration chamber.

To lyse tissue in a skin portion, the skin portion is aspirated into aspiration chamber 152 and, optionally, a set of piezoelectric transducers 156 that comprises less than all the piezoelectric transducers are excited to transmit ultrasound into the drawn up tissue. Optionally, the set of piezoelectric transducers 156 comprises contiguous transducers that are located along a portion of surface 157 and transmit ultrasound having an envelope and focal region similar to envelope 132 and focal region 133 shown in FIG. 3C.

In accordance with an embodiment of the invention, the envelope and focal region are rotated about axis 153 by rotating the location of the set of excited piezoelectric transducers in aspiration chamber 152. Changing which piezoelectric transducers 156 are comprised in the set rotates the set of excited piezoelectric transducers. Rotation of the ultrasound focal region operates to spatially homogenize the acoustic field that treats tissue aspirated into aspiration chamber 152 and can generate time dependent pressure gradients advantageous for treating tissue that are generally not evidenced by non-rotating focal patterns. In accordance with an embodiment of the invention, the angular velocity of rotation of the acoustic focal region is controlled so that time dependent stress generating pressure gradients generated by the rotation at a location in the tissue aspirated into chamber 152 matches relaxation times or resonant frequencies of the tissue. In some embodiments of the invention, different sets of piezoelectric transducers 156 are excited with different frequency AC voltage to generate a time dependent acoustic field in tissue aspirated into aspiration chamber 152.

The use of rotating acoustic fields and time dependent acoustic fields generated by exciting piezoelectric transducers with different frequency AC voltages is discussed in U.S. patent application Ser. No. 11870,445 to Andrey Rybyanets filed on Oct. 11, 2007, the disclosure of which is incorporated herein by reference.

In accordance with an embodiment of the invention, to moderate possible appearance of acoustic standing waves characterized by acoustic hotspots resulting from interference between transmitted acoustic energy and their reflections from surfaces inside aspiration chamber 152, transmitted acoustic energy is generated at frequencies that tend to obviate generation of acoustic standing waves in the chamber. For example, acoustic waves transmitted by a set of piezoelectric transducers 156 are generated at frequencies for which characteristic dimensions, e.g. diameter, of aspiration chamber 156, are not integer multiples of a half wavelength of the generated waves. Optionally, the characteristic dimension is equal to a multiple of a quarter wavelength of the transmitted ultrasound. In some embodiments of the invention, to moderate generating of standing wave hot spots, ultrasound is transmitted into tissue to be treated in bursts of less than about 20 wavelengths of the transmitted ultrasound. Optionally, the bursts comprise between about 10 and about 20 wavelengths.

It is noted, that whereas the aspiration chambers shown in FIGS. 3A-3D are circularly cylindrical, practice of the invention is not limited to circularly cylindrical aspiration chambers. For example, an aspiration chamber may have substantially any of various prismatic shapes or be a portion of a sphere. Furthermore, whereas transducers in the figures are shown rectangular, contiguous and arranged in two rows, various other configurations of transducers on inside surfaces of an aspiration chamber may be advantageous. For example, an aspiration chamber may be configured with a plurality of different groups of transducers, with each group separated from the other groups. Or an aspiration chamber may comprise more than two rows of transducers. Optionally, the transducers are rectangles having four equal sides or have shapes, for example hexagonal, other than rectangular.

In some embodiments of the invention, ultrasound is introduced through a region of skin into a body to treat tissue in the body at an angle, hereinafter a “tilt angle”, tilted away from the normal to the skin region. The entrance angle is tilted way from the normal to the skin region to moderate positive interference between the ultrasound introduced into the body and reflections of the ultrasound, for example from bone underlying the skin region, that might generate undesirable acoustic hot spots in tissue below the skin region or at the skin.

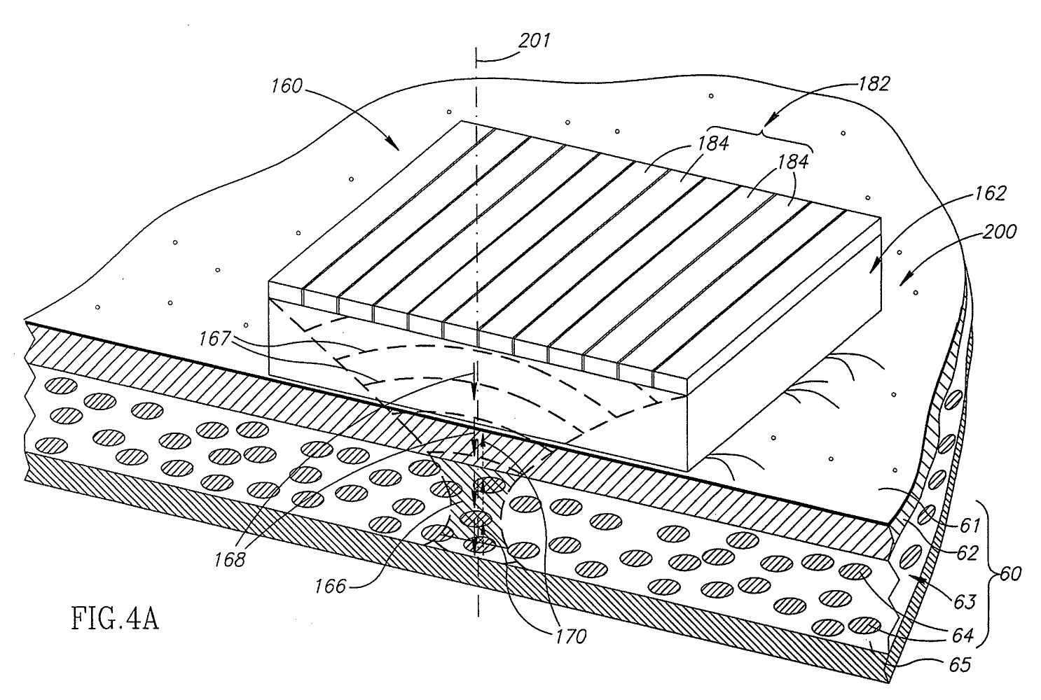

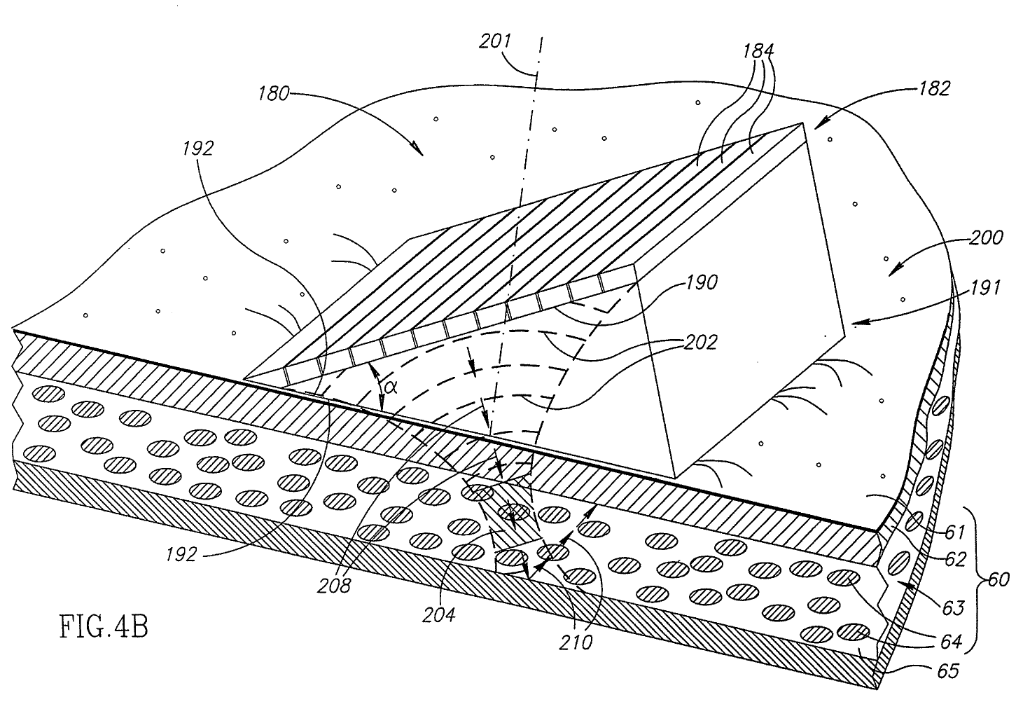

By way of example, FIG. 4A schematically illustrates an acoustic hot spot generated by an ultrasound apparatus 160 comprising a phased array 182 of piezoelectric transducers 184 that is excited to transmit ultrasound through a skin region 200 of a body (not shown) substantially along a normal 201 to the skin region. The transmitted ultrasound is assumed intended to lyse subcutaneous adipose tissue 64 located below the skin and over a region of bone 65. Optionally, piezoelectric transducers 184 are rectangular transducers and are coupled to a rectangular parallelepiped adapter 162 comprising a material in which, optionally, the speed of sound is substantially the same as that of body tissue. Optionally, the material is polyurethane, in which the speed of sound is about 1500 km/sec, substantially equal to that of adipose tissue.

Excited piezoelectric transducers 184 are schematically shown controlled to transmit ultrasound waves through skin region 200 that propagate parallel to normal 201 to the skin region so that it focuses in a focal region indicated by shading 166 below the skin having an extent substantially parallel to normal 201. Concentric arcs 167 represent waves of acoustic energy that propagate “downward” away from piezoelectric transducers 184 to enter the body and concentrate in focal region 166. Arrows 168 indicate direction of propagation of the acoustic energy. A portion, represented by dashed arrows 170, of transmitted acoustic energy 167 is reflected from bone 65 and passes back through focal region 166 to establish a standing wave pattern of acoustic energy that is characterized by an undesirably intense hot spot of acoustic energy in the focal region. For many applications such a hot spot can be undesirable and may for example, have intensity that burns and damages healthy skin.

FIG. 4B schematically shows a DEUS apparatus 180 configured to introduce ultrasound energy into adipose tissue shown in FIG. 4 so that it does not generate a hot spot, in accordance with an embodiment of the invention. DEUS apparatus 180, as in apparatus 160, optionally comprises a phased array 182 of piezoelectric transducers 184 for generating ultrasound. However, in DEUS 180, the piezoelectric transducers are coupled to a surface 190 of a wedge shaped adapter 191 having a wedge angle α between surface 190 and a contact surface 192 for coupling the apparatus to a patient's skin. In the figure, contact surface 192 is shown pressed to region 200 of the patient's skin 60 to couple DEUS 180 to the region to introduce ultrasound generated by phased array 182 through the skin and into the patient's body.

In accordance with an embodiment of the invention, wedge angle α provides for directing ultrasonic waves into the patient's skin tilted away from the normal to the skin region. Optionally, the tilt angle is equal to wedge angle α. In some embodiments of the invention, the wedge angle is between about 5° and about 45°. Optionally the wedge angle is between about 15° and about 35°

In FIG. 4B phased array 182 is schematically shown controlled to generate ultrasound waves represented by concentric arcs 202 that propagate toward skin region 200 and pass through the skin to focus in a focal volume below the skin indicated by shaded region 204. In accordance with an embodiment of the invention, the phased array transmits ultrasound 202 along a direction substantially perpendicular to surface 190. As a result, ultrasound 202 is tilted away from normal 201 to skin 200, in accordance with an embodiment of the invention, by a tilt angle equal to wedge angle α of wedge adapter 191. Direction of propagation of ultrasound waves 202 is represented by arrows 208.

After passing through focal region 204, a portion, represented by dashed arrows 210, of transmitted acoustic energy 202 is reflected from bone 65. However, unlike in FIG. 4A the reflected acoustic energy does not propagate back through focal volume 204 of the transmitted acoustic energy. The reflected acoustic energy is reflected at an angle of reflection substantially equal to tilt angle α and propagates toward the skin surface along a direction that misses the focal volume. As a result, interference between transmitted and reflected ultrasound does not generate an undesirable acoustic hot spot.

It is noted that whereas in FIG. 4B ultrasound 202 is transmitted along a direction perpendicular to surface 190, phased array 182 can of course be controlled to transmit ultrasound for lysing adipose tissue 64 along a direction that is not perpendicular to surface 190 to direct the ultrasound to enter skin region 200 at a non-zero tilt angle. And a non-zero tilt angle can be provided without having a wedge shaped adapter 191 to provide a non-zero tilt angle. For example, in accordance with an embodiment of the invention, phased array 182 shown in FIG. 4A is controlled to transmit ultrasound at a non-zero tilt angle into skin region 200.

In the description and claims of the present application, each of the verbs, “comprise” “include” and “have”, and conjugates thereof, are used to indicate that the object or objects of the verb are not necessarily an exhaustive listing of members, components, elements or parts of the subject or subjects of the verb.

The invention has been described with reference to embodiments thereof that are provided by way of example and are not intended to limit the scope of the invention. The described embodiments comprise different features, not all of which are required in all embodiments of the invention. Some embodiments of the invention utilize only some of the features or possible combinations of the features. Variations of embodiments of the described invention and embodiments of the invention comprising different combinations of features than those noted in the described embodiments will occur to persons of the art. The scope of the invention is limited only by the following claims.

1. Apparatus for lysing adipose tissue and/or cellulite comprising:
at least one transducer controllable to transmit ultrasound; and
an adapter configured to couple ultrasound transmitted by the at least one transducer to a region of a patient's skin comprising and/or overlaying a layer of adipose tissue and/or cellulite;
wherein the adapter orients the transmitted ultrasound relative to the layer of adipose tissue and/or cellulite so that it tends to propagate along a region of the layer.

2. Apparatus according to claim 2 and comprising a clamp having first and second panels which is operable to move the first panel towards the second panel to pinch up and clamp the region of skin and adipose and/or cellulite layer between the panels.

3. Apparatus according to claim 2 wherein the at least one transducer is located on the first panel and when controlled to transmit ultrasound, transmits the ultrasound in a direction toward the second panel.

4. Apparatus according to claim 3 and comprising an acoustic absorber is located on the second panel.

5. Apparatus according to claim 2 wherein the clamp is manually operable to move the first panel towards the second panel to pinch up and clamp the region of skin and adipose and/or cellulite layer.

6. Apparatus according to claim 2 and comprising a motor or actuator operable to move the first panel towards the second panel to pinch up and clamp the region of skin and adipose and/or cellulite layer.

7. Apparatus according to claim 1 wherein the at least one transducer is controllable to transmit ultrasound in a direction substantially parallel to a portion of the adipose layer and/or cellulite clamped between the first and second panels.

8. Apparatus according to claim 7 wherein the at least one transducer is controllable to focus the ultrasound in a focal volume located in the portion of the adipose and/or cellulite layer.

9. Apparatus according to claim 8 wherein the at least one transducer is controllable to translate the focal volume in a direction substantially perpendicular to a direction along which the clamp pinches up the region of skin and substantially perpendicular to a direction along which the at least one transducer transmits the ultrasound.

10. Apparatus according to claim 8 wherein the at least one transducer is controllable to focus the ultrasound in a focal volume that has an extent substantially equal to an extent of the at least one transducer in a direction substantially perpendicular to a direction along which the clamp pinches up the region of skin and substantially perpendicular to direction along which the at least one transducer transmits the ultrasound.

11. Apparatus according to claim 1 wherein the adapter comprises an aspiration chamber into which the region of skin and the adipose and/or cellulite layer can be drawn up by aspirating air from the chamber.

12. Apparatus according to claim 11 wherein the at least one transducer is located on an internal surface of the aspiration chamber substantially parallel to a direction along which the region of skin and adipose and/or cellulite layer is drawn up into the aspiration chamber.

13. Apparatus according to claim 11 wherein the at least one transducer is controllable to transmit ultrasound in a direction substantially parallel to a portion of the adipose and/or cellulite layer drawn up into the aspiration chamber.

14. Apparatus according to claim 13 wherein the at least one transducer is controllable to focus the ultrasound in a focal volume located in the portion of the adipose and/or cellulite layer.

15. Apparatus according to claim 14 wherein the at least one transducer is controllable to move the focal volume.

16. Apparatus according to claim 15 wherein the at least one transducer is controllable to rotate the focal volume around an axis substantially parallel to the direction along which the region of skin and adipose tissue and/or cellulite is drawn up into the aspiration chamber.

17. Apparatus according to claim 11 wherein the aspiration chamber is cylindrical.

18. Apparatus for lysing adipose tissue and/or cellulite in a patient, the apparatus comprising:
at least one transducer controllable to transmit ultrasound; and
a wedge shaped transducer having a first surface on which the at least one transducer is located and a second surface through which ultrasound transmitted by the at least one transducer enters the patient's skin;
wherein the first and second surfaces are angled with respect to each other by a wedge angle.

19. Apparatus according to claim 18 wherein the wedge angle is between about 5° and about 45°.

20. Apparatus according to claim 18 wherein the wedge angle is between about 15° and about 35°.

21. Apparatus according to claim 1 wherein the at least one transducer comprises a plurality of transducers configured in a phased array of transducers.

22. A method of treating adipose tissue and/or cellulite in a patient, the method comprising:
drawing a region of the patient's skin and a layer of adipose tissue and/or cellulite comprised in the skin and/or overlaid by the skin away from the body; and
transmitting ultrasound through the drawn away skin region and adipose tissue so that it propagates through a portion of the drawn away adipose and/or cellulite layer along a direction substantially parallel to the layer.

23. A method according too claim 22 wherein transmitting ultrasound along a direction substantially parallel to the layer comprises transmitting the ultrasound along a direction substantially perpendicular to a direction along which the skin region is drawn away from the body.

24. A method according to claim 22 wherein transmitting comprises focusing the ultrasound in a focal volume located in the portion of the adipose and/or cellulite layer.

25. A method according to claim 24 and comprising moving the focal volume.

26. A method according to claim 25 wherein moving comprises translating the focal volume along a direction substantially perpendicular to the direction along which the region of skin and adipose tissue and/or cellulite is drawn away from the body.

27. A method according to claim 25 wherein moving comprises rotating the focal volume along a direction substantially parallel to the direction along which the region of skin and adipose tissue and/or cellulite is drawn away from the body.
